(19) United States
(12) Patent Application Publication
(10) Pub. No.: US 2018/0368814 A1
(43) Pub. Date: Dec. 27, 2018

(54) INTELLIGENT APPLICATION FOR AN ABSORBENT MULTIFUNCTIONAL DISPOSABLE HYGIENE APPARATUS, SYSTEM AND METHODS EMPLOYED THEREOF

(71) Applicant: Parag R. Kudtarkar, Mumbai (IN)

(72) Inventor: Parag R. Kudtarkar, Mumbai (IN)

(21) Appl. No.: 15/775,843

(22) PCT Filed: Jun. 3, 2016
(86) PCT No.: PCT/IB2016/053263

§ 371 Date: May 14, 2018

(30) Foreign Application Priority Data
Feb. 26, 2016 (IN) 201621006750

(51) Int. Cl. A61B 10/00; A61B 5/00; A61B 5/01; A61B 5/20; A61B 5/145; G16H 80/00; G01N 33/49

(52) U.S. Cl. CPC A61B 10/0012; A61B 2010/0006; A61B 5/6802; A61B 5/01; A61B 5/7435; A61B 5/746; A61B 5/208; A61B 5/4318; A61B 5/6843; A61B 5/14507; G16H 80/00; G01N 33/492; A61B 2562/08; A61B 2010/0019; A61B 5/0022

(57) ABSTRACT

Exemplary embodiments of the present disclosure are directed towards an electronic module positioned in a multifunctional disposable hygiene apparatus of a user, wherein the electronic module comprises of: a plurality of sensors enabled to sense a plurality of parameters of a discharge and read a pressure generated due to a seating pattern of the user; and an embedded dipstick configured to conduct a test of a plurality of blood parameters from the discharge; a network device of a user comprising an application and in communication with the multifunctional disposable hygiene apparatus, wherein the application is configured to receive a data comprising data comprising the plurality of parameters of a discharge and pressure generated due to the seating pattern of the user and the plurality of blood parameters from the discharge from the electronic module; a remote data centre in communication with the user's network device configured to store and process the data received from the user's network device and transmitting notifications to the user's network device in response to processing the data; and a medical service provider's network device in communication with the user's data centre and the user's network device configured to allow the medical service provider to view the data of the user.

### TECHNICAL FIELD

The present disclosure generally relates to the field of absorbent personal care hygiene products. More particularly, the present disclosure relates to an intelligent, multifunctional absorbent pad.

### BACKGROUND

Absorbent pads are expected to work on health, safety, convenience along with ecosystem compatibility and non-invasiveness. There has always been an ambiguity in the intervals required for change of absorbent pad keeping in mind the hygiene aspects. Infrequent changes increase the chances of vaginal infections in women and other urinary tract infections in medicating adults having undergone post-operative therapy etc. These issues may lead to malignancy if left untreated. Hygiene is important in women of reproductive age in order to abet sexually transmitted diseases. It is also essential in the aging population, adults and kids using absorbent pads. Menstrual cycle is subjective and differs from one woman to another. Along with menstruation there also exists a need for a woman to be alerted about the ovulation date, fertility period, testing of blood parameters and also the frequency of change of sanitary absorbent pad.

The adults who are undergoing any nursing or people who have come of age suffering with incontinence need to know the frequency and time intervals required to change the absorbent material. It is also challenging for parents and guardians to realise the intervals required to change the absorbent pad in kids. The existing sanitary or absorbent pads do not have the capability to judge the physiological indicators.

Women's health and hygiene is influenced to a large extent by societal norms, culture and personal attention. A woman's experience with genital hygiene involves various stages; from infancy to teens to motherhood to aging which involves menstruation, recovery from vagina surgery, post birth bleeding, post abortive care to name a few where vaginal discharge or bleeding is experienced. Be it a homemaker, a working woman or a military woman dealing with combat activities, contemporary woman has been progressive. A busy schedule involving multitasking has prompted the consumer industry to work on the necessities of absorbing large volumes of blood in a short span of time and ensuring the absorptivity at the right place preventing backflow of blood.

In the light of aforementioned discussion there exists a need to have a sanitary napkin embedded with a device which would alert the user on the above mentioned parameters with accuracy and respecting one's privacy.

### BRIEF SUMMARY

The following presents a simplified summary of the disclosure in order to provide a basic understanding to the reader. This summary is not an extensive overview of the disclosure and it does not identify key/critical elements of the invention or delineate the scope of the invention. Its sole purpose is to present some concepts disclosed herein in a simplified form as a prelude to the more detailed description that is presented later.

Exemplary embodiment of the present disclosure is directed towards an intelligent application for an absorbent multifunctional disposable hygiene apparatus, system and methods employed thereof.

Another exemplary aspect of the present subject matter is directed towards an intelligent application for an absorbent multifunctional disposable hygiene apparatus, system and methods employed thereof comprising of an electronic monitoring system with a quick response code (QRC); a plurality of sensors enabled to sense the colour, odour, temperature of the discharge and the pressure generated due to seating pattern of the user; a flexible printed circuit board (PCB); an embedded dipstick enabled to conduct the test of the blood parameters from the discharge; a microcontroller with a memory and programmable input and output peripherals.

Another exemplary aspect of the present subject matter is directed towards an intelligent absorbent personal care hygiene apparatus comprising of a short term network enabled to communicate to the user's hand held network device through a set up via QRC.

Another exemplary aspect of the present subject matter is directed towards a registration logic wherein the user is enabled to be registered through not limiting to one of the social networking accounts and the user is further enabled to choose and display the menstrual cycle regularity and irregularity, the first date of the last menstrual cycle and the average days in the menstrual cycle of the user.

Another exemplary aspect of the present subject matter is directed towards enabling the user to be alerted regarding the fullness of the intelligent absorbent personal care hygiene apparatus with the discharge and also alerting the user about the scheduled date for the next menstrual cycle through an alert logic.

Another exemplary aspect of the present subject matter is directed towards a graphical representation wherein the readings of the parameters in the selected month are displayed.

Another exemplary aspect of the present subject matter is directed towards the choice of settings wherein the user is enabled to select one of personal settings, cycle settings or an alert settings depending upon the requirement.

### BRIEF DESCRIPTION OF DRAWINGS

Other objects and advantages of the present invention will become apparent to those skilled in the art upon reading the following detailed description of the preferred embodiments, in conjunction with the accompanying drawings, wherein like reference numerals have been used to designate like elements, and wherein:

FIG. 1 is a diagram depicting a data processing environment of an absorbent multifunctional disposable hygiene apparatus, according to an exemplary embodiment of the present disclosure.

FIG. 2 is a diagrammatic representation of an electronic module embedded in the absorbent multifunctional disposable hygiene apparatus, according to an exemplary embodiment of the present disclosure.

FIG. 3A is a flow diagram representing the method of functioning of a data processing environment of an absorbent multifunctional disposable hygiene apparatus, according to an exemplary embodiment of the present disclosure.

FIG. 3B is a flow diagram representing the method of working of the intelligent application absorbent multifunctional disposable hygiene apparatus, according to an exemplary embodiment of the present disclosure.

FIG. 4 is a diagram representing a welcome logic of the intelligent application for absorbent multifunctional disposable hygiene apparatus, according to an exemplary embodiment of the present disclosure

FIG. 5 is a diagram representing user registration logic for the intelligent application for an absorbent multifunctional disposable hygiene apparatus, according to an exemplary embodiment of the present disclosure.

FIG. 6 is a diagram representing connectivity logic for an absorbent multifunctional disposable hygiene apparatus, according to an exemplary embodiment of the present disclosure.

FIG. 7 is a diagram representing view logic for an absorbent multifunctional disposable hygiene apparatus, according to an exemplary embodiment of the present disclosure.

FIG. 8 is a diagram representing an alert logic for an absorbent multifunctional disposable hygiene apparatus, according to an exemplary embodiment of the present disclosure.

FIG. 9 is a diagram representing history logic for an absorbent multifunctional disposable hygiene apparatus, according to an exemplary embodiment of the present disclosure.

FIG. 10 is a diagram representing settings logic for an absorbent multifunctional disposable hygiene apparatus, according to an exemplary embodiment of the present disclosure.

### DETAILED DESCRIPTION

It is to be understood that the present disclosure is not limited in its application to the details of construction and the arrangement of components set forth in the following description or illustrated in the drawings. The present disclosure is capable of other embodiments and of being practiced or of being carried out in various ways. Also, it is to be understood that the phraseology and terminology used herein is for the purpose of description and should not be regarded as limiting.

The use of “including”, “comprising” or “having” and variations thereof herein is meant to encompass the items listed thereafter and equivalents thereof as well as additional items. The terms “a” and “an” herein do not denote a limitation of quantity, but rather denote the presence of at least one of the referenced item. Further, the use of terms “first”, “second”, and “third”, and the like, herein do not denote any order, quantity, or importance, but rather are used to distinguish one element from another.

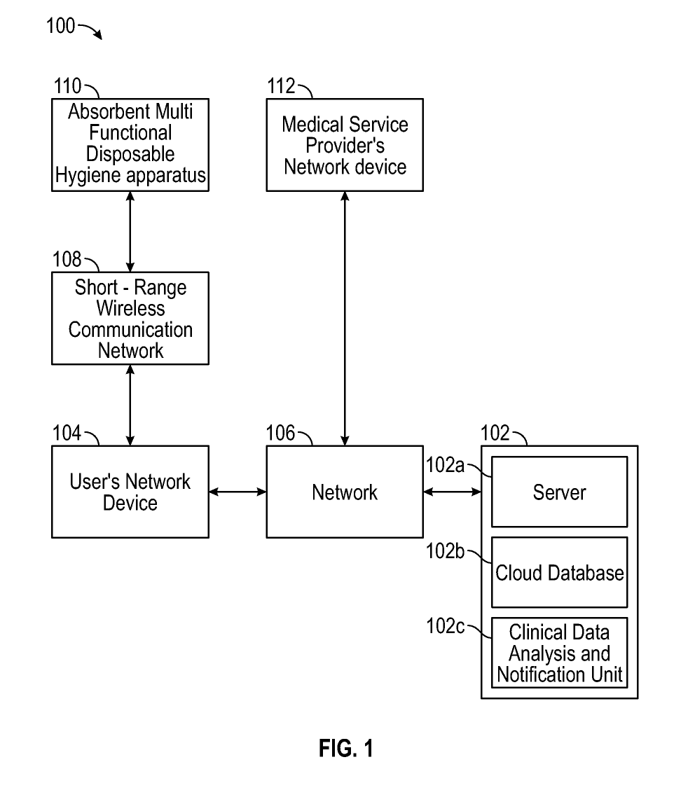

Referring to FIG. 1 is a diagram 100 depicting a data processing environment of an absorbent multifunctional disposable hygiene apparatus, 110, according to an exemplary embodiment of the present disclosure. The data processing environment includes an absorbent multifunctional disposable hygiene apparatus 110 (herein after referred as hygiene apparatus 110) embedded with electronic module. The electronic module configured to establish a secured and authenticated communication link between the hygiene apparatus 110 and a user's network device 104 through a short range wireless network 108. The user's network device 104 is enabled to display the readings obtained from the hygiene apparatus 110 through a short range communication network 108. The short range wireless network 108 may include but not limited to a Zigbee, Bluetooth, Infrared, Wifi, and NFC. The electronic module further configured to collect the data from the user and transmit to the user's network device 104. The scope of the components involved in the apparatus 110 is extendable to adult absorbent pad, kid absorbent pad and the like without limiting the scope of the disclosure. The user's network device 104 may be extendable to smart phones, smart watches and to non-hand held devices like laptops, tabs and the like without limiting the scope of the disclosure.

The data processing environment includes a data server system 102 comprising a server 102a and a cloud database 102b for routing and storing the data. The server system 102 also includes a data analysis and notification unit 102c which is configured to analyze and process the data pushed by the user's network device 104 and further transmits notifications to the user's network device 104 in response to processing and analyzing the data. The data processing environment may further include a medical service provider's network device 112. The user's network device 104, the medical service provider's network device 112 and the server system 102 are connected over a network 106. The medical service provider's network device 112 enables the medical service provider to analyze the results shared from the user's network device 104. The medical service provider may be authorized by the user of the apparatus 110 to access the results to relatively alert the user if any abnormalities are found in the results communicated via a network to the user's network device 104. The network 106 may include, but not limited to, an Ethernet, a local area network (LAN), or a wide area network (WAN), e.g., the Internet, or a combination of networks.

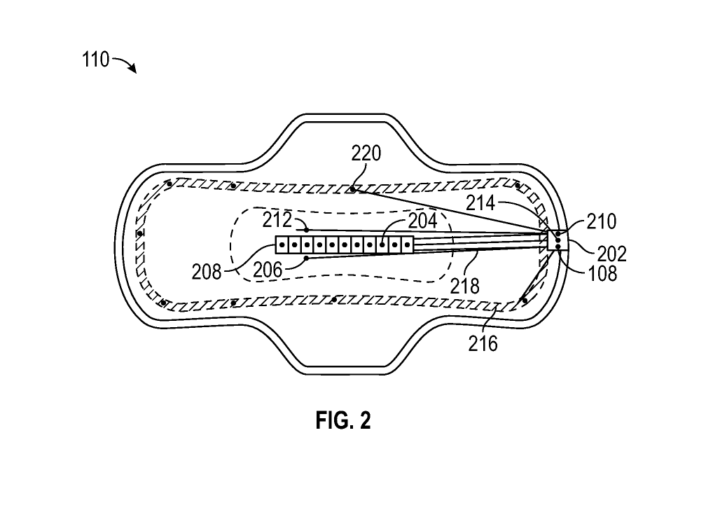

Referring to FIG. 2 is a diagrammatic representation 110 of an electronic module embedded the hygiene apparatus, according to an exemplary embodiment of the present disclosure. The electronic module comprises of a flexible printed circuit board (PCB) 202, which is enabled to form a link between conductive components and non-conductive components of the electronic module. The PCB 202 is enabled to be flexible so that it does not interfere with the twists and turns associated with the hygiene apparatus 110. The flexibility also ensures that the components do not hurt the user through protrusion. The electronic monitoring system further comprises multiple sensors. For example, sensors including the colour sensors 204 which are enabled to monitor the colour of the discharge. The discharge could be blood as in a menstrual cycle or urine or any other post-operative discharge and the like. The number of colour sensors 204 could be many wherein each of the sensors would denote the reading of each of the parameters either in blood and/or urine and/or as the case may be.

Each of the colour sensors 204 are placed in each grid of the dipstick 208. The colour change is generated at the center of the hygiene apparatus 110 which may be enabled to be like a dipstick 208, wherein the flow of the fluid is high. The flow of the discharge gets first absorbed in the center and further goes down the hygiene apparatus 110 and then comes up. When the discharge falls on the dipstick 208 then the colour sensors 204 located right below the dipstick 208 change their colour according to the discharge. The colour changes are read periodically in the duration of the apparatus 110 being utilised. Each of the colour grids may be in the form of a strip may change the colour depending upon the chemical parameters it has been designed to take a reading. For example; Urea, Creatinine, Sodium, Potassium, Calcium, Phosphate, Albumin, potential RISKS like Cholesterol, Triglyceride or Urate, Parathyroid Hormone, serum ferritin, complete blood picture, Hepatitis, pregnancy test and the like without limiting the scope of the disclosure. Each of the parameter would reflect each of the colors thus enabling the user to know of any abnormalities associated with the colour change.

The odour sensor 206 is enabled to perceive the foul odour generated as a result of not changing the hygiene apparatus 110 at regular intervals. The odour sensor 206 is specifically useful in case of absorbent pad for kids where it can sense whether the kid user has urinated or has pooped. Since the poop would be solid hence it is the odour sensor 206 which can sensitize that the hygiene apparatus 110 needs to be changed. A temperature sensor 212 is enabled to perceive the temperature of the body. This mechanism is useful to denote in case there is a temperature rise in of adults, kids or in a menstruating user wherein the temperature of the body undergoes a change during the cycle. A pressure sensor 216 is enabled to sensitize the pressure irregularities on the hygiene apparatus 110 generated due to the seating pattern of the user. The pressure sensor 216 is placed all over the grid of the absorbent apparatus 110 at the peripheries. The irregularities in the pressure may enable the discharge to flow out of the hygiene apparatus 110 thus leading to discomfort to the user. Further a microcontroller 210 is enabled to perform as a single integrated circuit comprising of a processor core and a programmable input and output peripherals. The microcontroller 210 is used to display or manipulate the data as per the requirement. The short range wireless communication network 108 acts as a communication between the user's network handheld device 104 and the hygiene apparatus 110. A battery 214 is enabled to act as a source of energy to the electronic module. The PCB 202 contains the short range wireless communication network 108, the battery 214 and the microcontroller 210 embedded in it. The PCB 202 is located at an extreme corner of the apparatus 110, and is connected to all the sensors as in colour sensor 204, odour sensor 206, temperature sensor 212 and pressure sensors 216 in order to send the appropriate readings and signals to be registered. A liquid sensor 220 is configured in the rim accommodating the pressure sensors 216. The liquid sensor 220 is enabled to sensitize the absorption of the liquid as in the discharge and once the apparatus 110 exceeds in the limit of absorbing the liquid the liquid sensor 220 registers the readings as it is connected to the PCB 202. The registered readings enable the user's network device 104 to generate an alert with respect to the fullness of the apparatus 110.

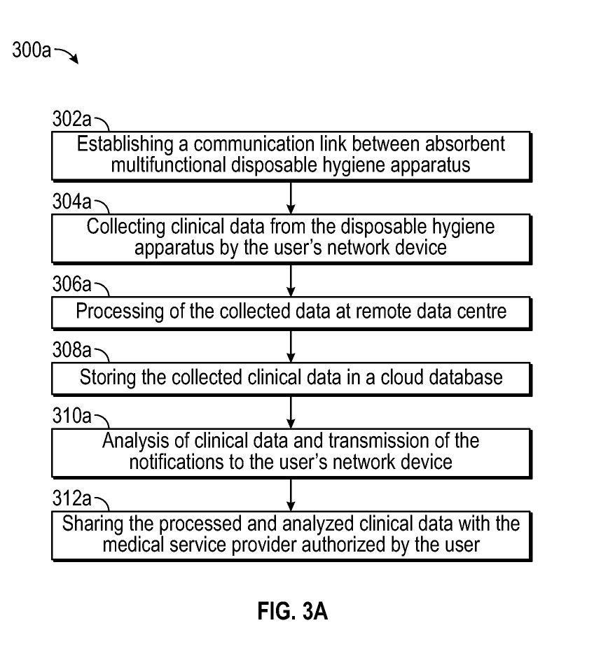

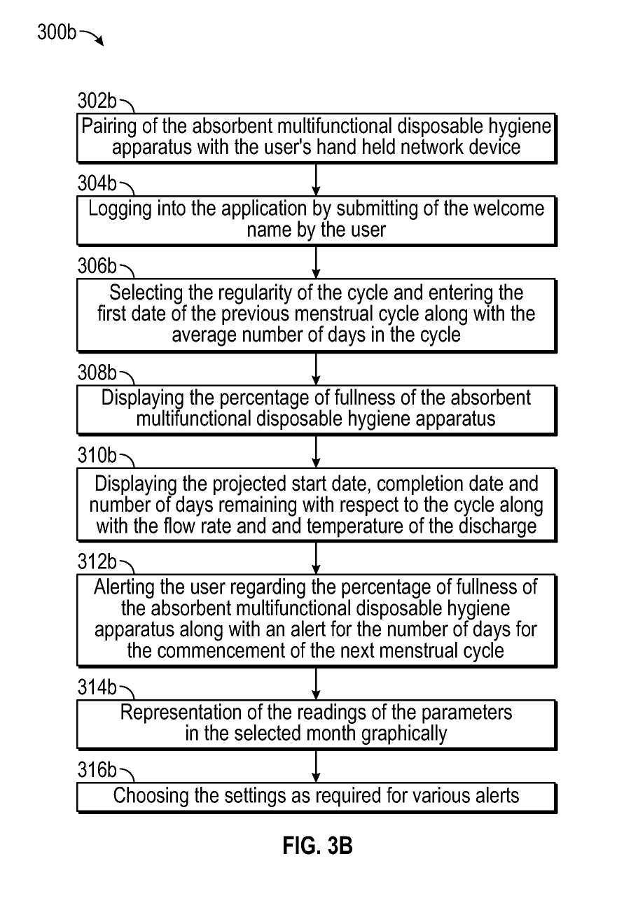

FIG. 3A is a flow diagram 300a, representing a method of functioning of a data processing environment of an absorbent multifunctional disposable hygiene apparatus 110. The method commences at step 301 by establishing a communication link between absorbent multifunctional disposable hygiene apparatus 110, a user's network device 104 followed by collecting data from the disposable hygiene apparatus by the user's network device 104 at step 303. Further the processing of the collected data through an established network is done at step 305. Further at step 307 the collected data is stored in a cloud database. Transmission of the notifications of the user's network device 104 to the data analysis and notification unit is done at step 309. Notifying the medical service provider and the user through their user devices about the analysis is done at step 311.

Referring to FIG. 3B is a flow diagram 300b representing the method of working of the hygiene apparatus 110, according to an exemplary embodiment of the present disclosure. The method starts at step 302 with the pairing of the absorbent multifunctional disposable hygiene apparatus 110 with the user's handheld network device 104. Further the user logs into the application by submitting one's welcome name as in step 304. At step 306, selection of the regularity of the cycle and entering the first date of the previous menstrual cycle along with the average number of days in the cycle is done. Further at step 308, the percentage of fullness of the hygiene apparatus 110 is displayed. The projected start date, completion date and number of days remaining with respect to the cycle along with the flow rate and temperature of the discharge are displayed at step 310. The user is alerted regarding the percentage of fullness of the absorbent multifunctional disposable hygiene apparatus 110 along with an alert for the number of days for the commencement of the next menstrual cycle at step 312. A graphical representation is displayed of the readings of the parameters in the selected month at step 314. Finally at step 316 the choice of the settings is done as per the requirement of the user.

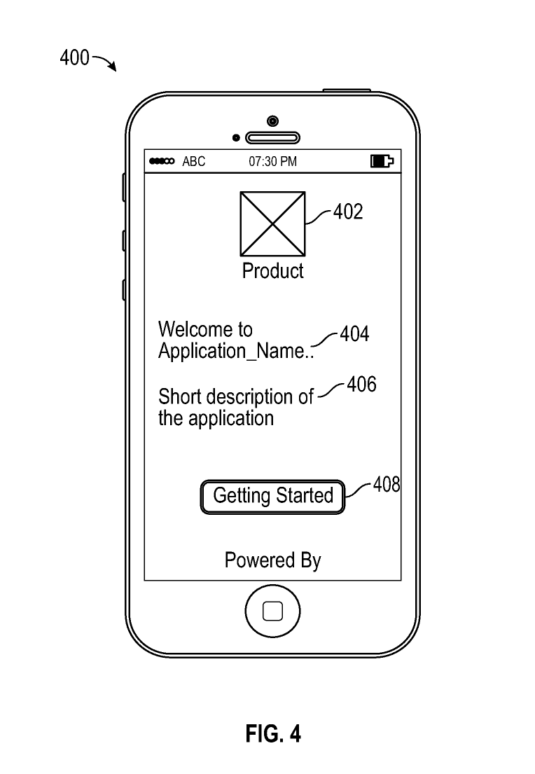

Referring to FIG. 4 is a diagrammatic representation 400 of a welcome logic of the intelligent application for an absorbent multifunctional disposable hygiene apparatus, according to an exemplary embodiment of the present disclosure. The user's network device 104 is enabled to display the absorbent multifunctional disposable hygiene apparatus (referred to as a product in FIG. 402. At 404 the user is enabled to access the application with a welcome sign followed by the name of the application. A short description of the application 406 is provided. The get started button 408 enables the user to go ahead with the further process in the application.

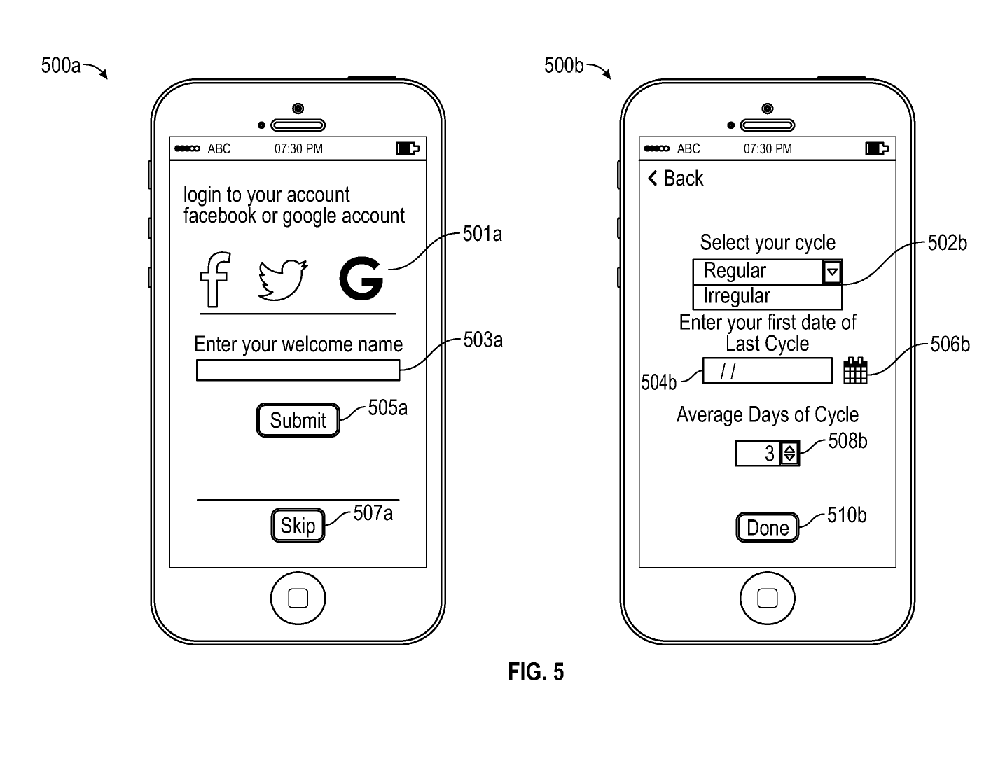

Referring to FIG. 5A-5B are a diagrammatic representations 500a and 500b of user registration logic of the intelligent application for an absorbent multifunctional disposable hygiene apparatus, according to an exemplary embodiment of the present disclosure. As shown in FIG. 5A a process for logging into the application with the help of the user's account using one of the social networking accounts 501a, without limiting the scope of the disclosure. This is helpful in case of the user losing one's network device or if the user chooses to change the network device and synchronize the data accordingly. The user may be enabled to feed a welcome name 503a as per one's choice. The welcome name can be submitted as in the field 505a. In case the user is not interested to save the data then the skip option as in 507a may be enabled. As shown in FIG. 5B the applications enable the user to access the further parameters displayed on the user's network device 104 wherein the user may choose to fill in the details which would enable the accurate functioning of the application. Multiple questions displayed by the application may include a selection of the regularity of the menstrual cycle 502b which can be done by enabling the user to choose whether one's menstrual cycle is regular or irregular. The first date of the previous menstrual cycle can be entered as in 504b. The dates can be automatically chosen by clicking at 506b wherein the calendar is displayed. The average number of days involved in the user's menstrual cycle can be entered as in 508b. It is as per this feed the further calculations will be enabled in the application accordingly. Upon entering the appropriate details a done button 510b can be pressed for the details to get stored.

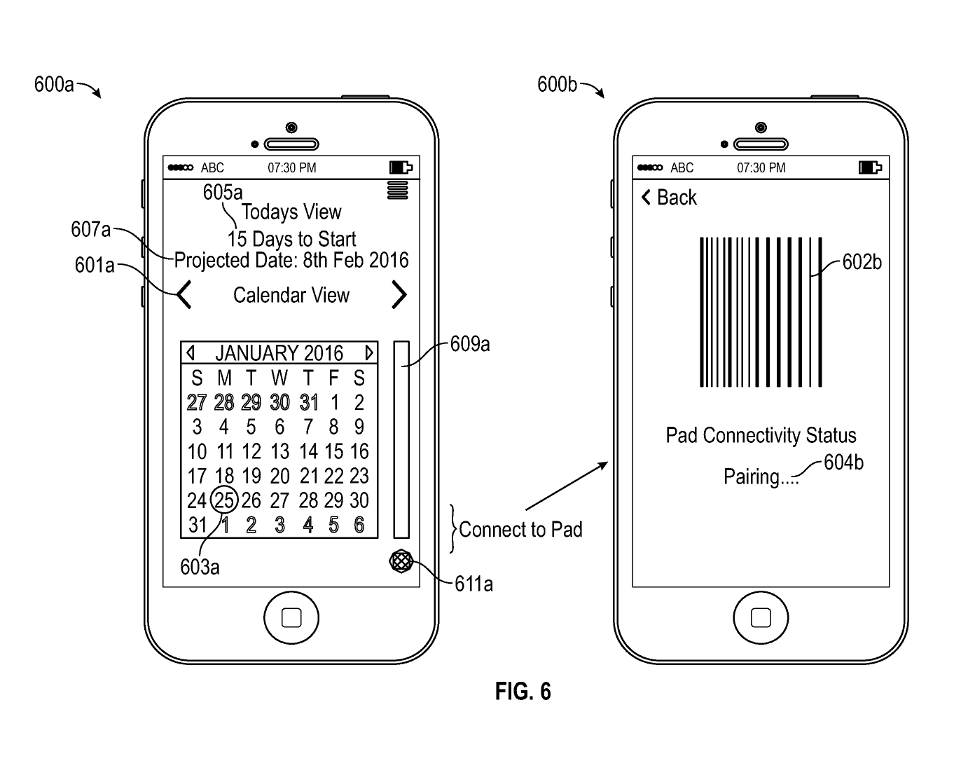

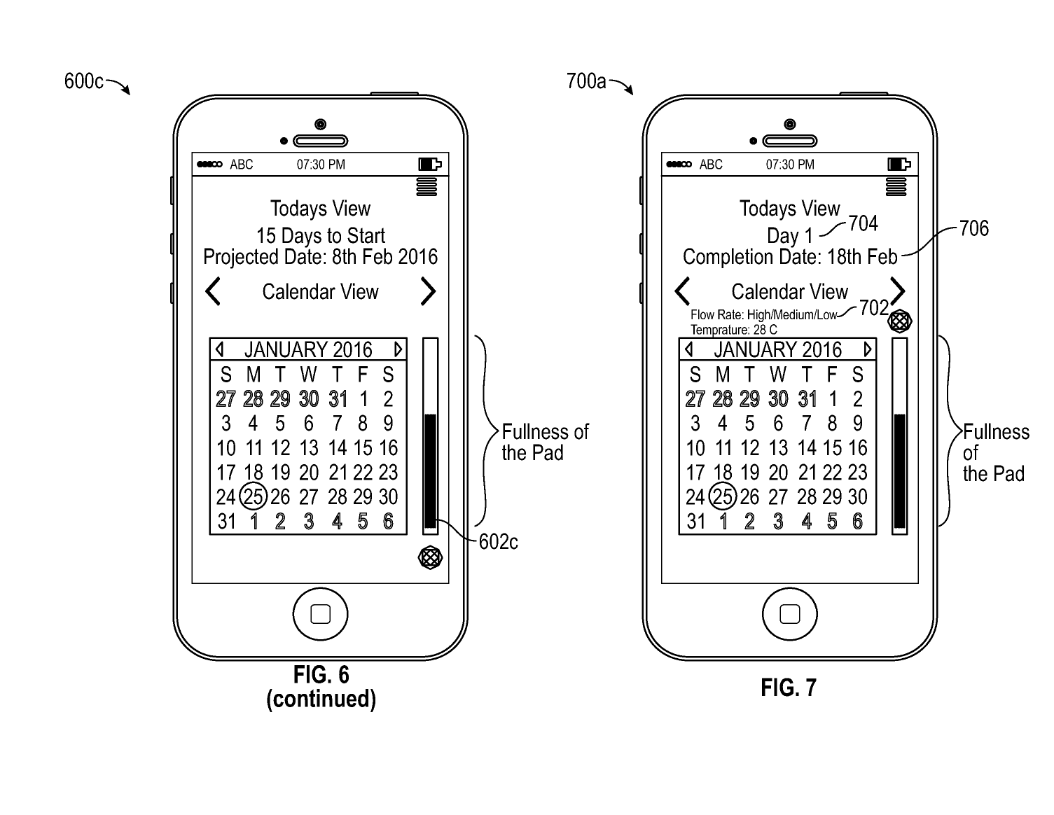

Referring FIG. 6A-6B are diagrammatic representations 600a and 600b of connectivity logic for the hygiene apparatus, according to an exemplary embodiment of the present disclosure. As shown in 600a, an option to view the calendar in 601a is provided by the application. The calendar view displays the calendar with the view of the name of that particular month followed by the year. The display also includes the first alphabet of the days in a week along with the dates in a month. For example, the current date is displayed with a shade in 603a. The number of days left out for the commencement of the menstrual cycle can be displayed at 605a. The projected date for the menstrual cycle to commence is displayed in 607a. The level of discharge absorbed in the absorbent multifunctional disposable hygiene apparatus 110 is displayed as an empty box 609a as the user may not be using the apparatus. The field 611a would enable the user to choose whether one wants to connect the hygiene apparatus 110 with the user's network device 104. By pressing the icon the connectivity starts else the user may choose not to connect with the device. The interface shown in 600a is displayed once the usage of the hygiene apparatus 110 starts. A quick response code (QRC) 602b is unique to each of the hygiene apparatus 110 and is linked to the application in the user's network device 104. The QRC 602b is marked on the packet used to pack the hygiene apparatus 110. The pairing between the hygiene apparatus 110 and the user's network device 104 starts with the display of the pairing status at 604b. The QRC 602b is unique for each of the hygiene apparatus and has an ID and a password embedded in it. The bar code as in 602b is a camera view wherein it automatically scans the QRC 602b and connects to the bluetooth and the pairing starts as shown in 604b. In FIG. 600c the fullness of the hygiene apparatus 110 is displayed wherein the user is enabled to see the level of the fullness at 602c.

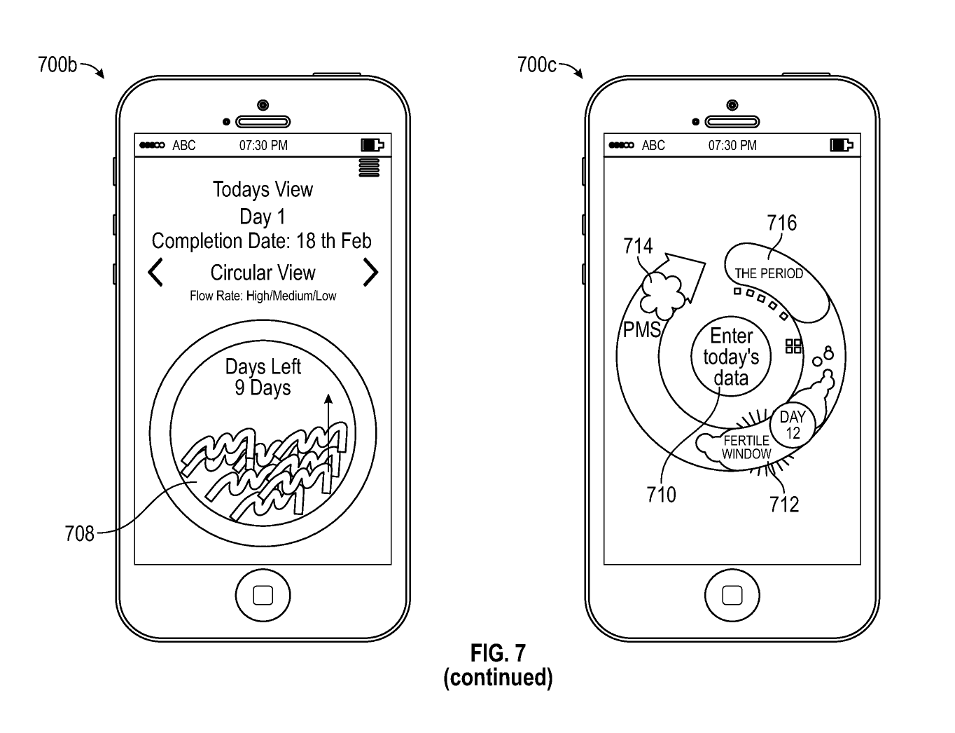

Referring to FIG. 7A-7B are diagrammatic representations 700a and 700b of view logic for a hygiene apparatus 110, according to an exemplary embodiment of the present disclosure. The calendar view is represented as 700a. The intensity of the flow of the discharge along with the body temperature is represented as 702. This enables the user to have an idea about his existing health condition. The number of the day into the cycle is displayed as 704 and the projected date of completion of the cycle is displayed as 706. The number of days left over for the completion of the menstrual cycle is calculated based on the ongoing day of the cycle and is displayed in a circular view 700b wherein the colour in the circle 708 gets rising as and when the intelligent absorbent hygiene apparatus gets filled with the discharge which could be blood, urine and the like. FIG. 702c is a detailed circular calendar view of the cycle and other details associated with it. Any specific data required to be entered for the data can be done by the user as displayed in 710. The fertile window 712 would enable the user to know the days from when she would be fertile. Premenstrual syndrome (PMS) 714 enables the user to have emotional swings idea which is normally observed in women during PMS. The period (menstrual cycle) 716 enables the user to note the dates of the cycle. The circular calendar view as in 702c enables the user to have an accurate display of only those dates which are required for the user unlike the calendar view which would display all the dates.

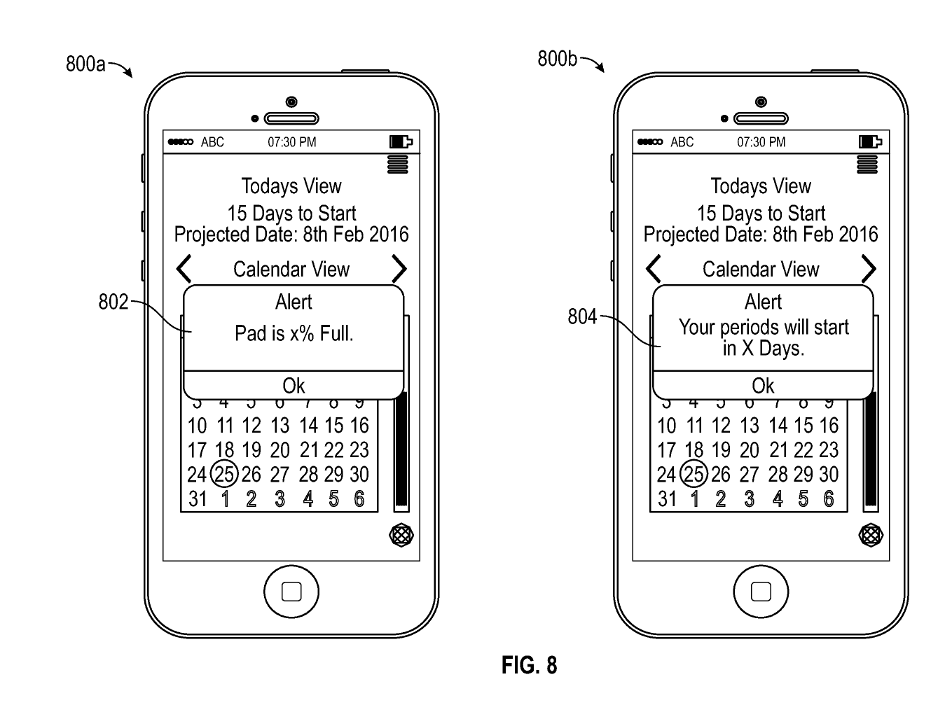

Referring to FIG. 8A-8B are a diagrammatic representations 800a and 800b of an alert logic for a hygiene apparatus, according to an exemplary embodiment of the present disclosure. An alert is represented as 802 wherein the percentage of the fullness of the absorbent multifunctional disposable hygiene apparatus 110 is displayed. Another alert regarding the number of days remaining for the next menstrual cycle is also displayed as 804. The flow rate of the discharge and its absorptivity depends upon the activities which the user is involved. For example the seating pattern. This may enable the discharge to flow out or leak out of the acceptable area in the hygiene apparatus 110 and reach the rim. Once this happens then also an alert is generated. These alerts enable the user to be prepared and deal for the contingencies involved.

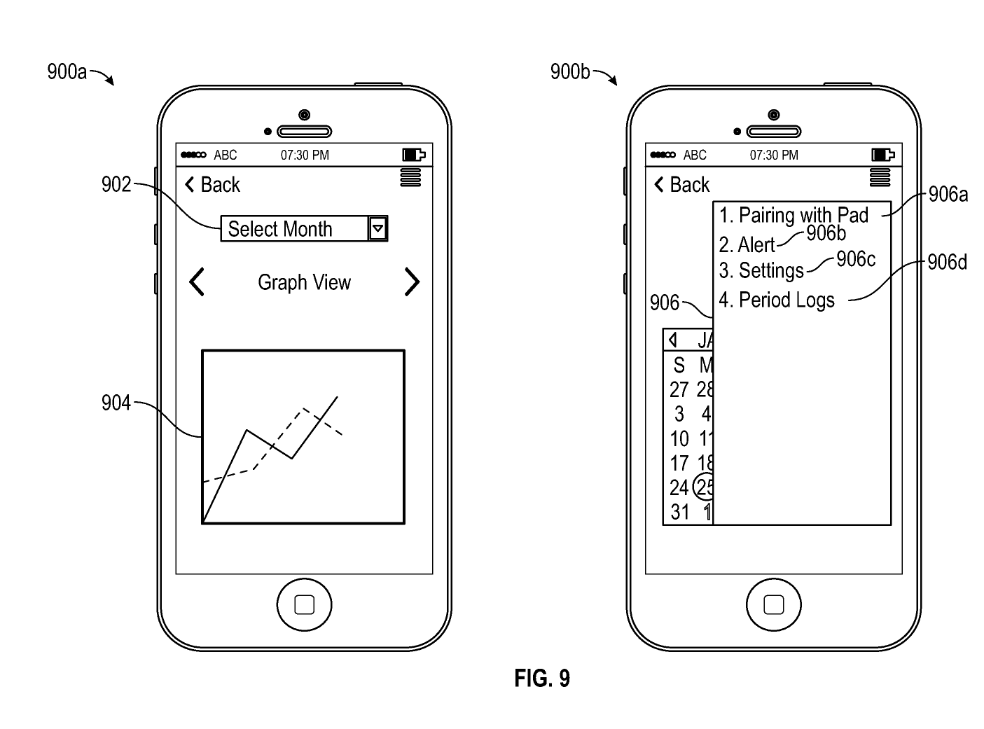

Referring to FIG. 9A-9B are diagrammatic representations 900a and 900b of history logic for a hygiene apparatus, according to an exemplary embodiment of the present disclosure. At 902 the user is enabled to select the month which one wishes to see or retrieve the data of. The graphical representation 904 of the various parameters calculated for a selected month is displayed with different colors for different parameters enabling the user to study the health condition. The parameters represented graphically 904 may include, but not limited to, the number of hygiene apparatus 110 used in the cycle, the flow rate of the discharge more particularly, but not limiting to, a menstrual discharge and the temperature. At 906 a drop down menu to view the various options is displayed. The pairing option with the hygiene apparatus 110 is displayed as 906a, the alert option is enabled at 906b, and the setting option at 906c and the period logs option at 906d.

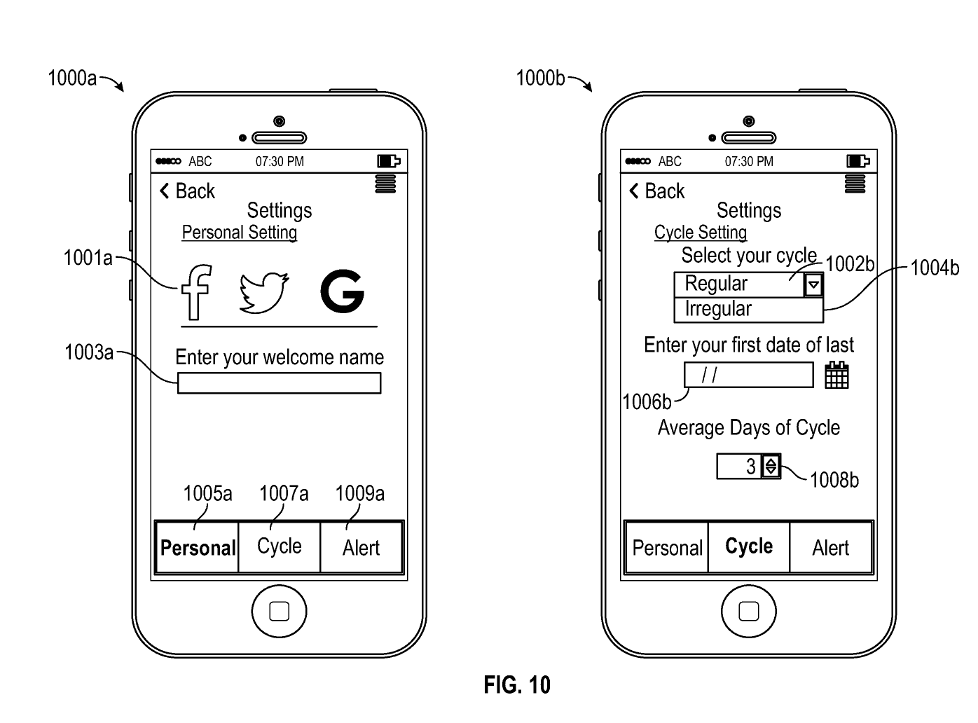

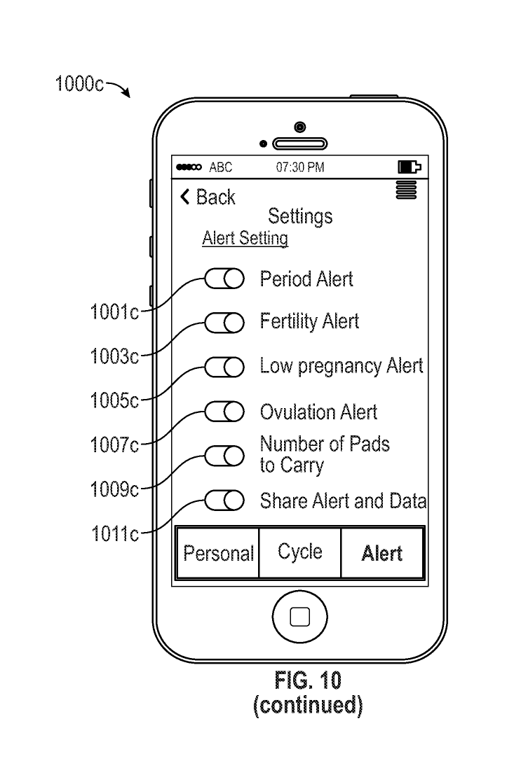

Referring to FIG. 10A-10C are diagrammatic representations 1000a, 1000b and 1000c of settings logic for a hygiene apparatus, according to an exemplary embodiment of the present disclosure. The Personal settings 1000a interface includes a welcome name 503a that can be submitted as in 1003a, logging in the application by accessing various social networking sites accounts 1001a. The type of settings required can be chosen by clicking on to the personal button 1005a, cycle 1007a and an alert 1009a. The cycle settings 1000b involve the display of the regularity of the cycle as a regular cycle 1002b or an irregular cycle as 1004b. The irregularity in the cycle would enable the medical service provider to alert the user regarding any prevailing gynecological or any other complications. The first date of the previous cycle can be entered as in 1006b and the average numbers of days experienced by the user as a cycle can be set as in 1008b.

The alert settings 1000c enables the user to choose an option of period alert 1001c enabling the user to create an alert regarding the menstrual cycle. A fertility alert 1003c enables the user to create an alert regarding those days of the month where the user may be fertile. A low pregnancy alert 1005c enables the user to know those days where the probability getting pregnant is on the lower side. This would enable the user to decide whether she can be subjected to safer intercourse with or without contraception depending upon the requirement. An ovulation alert 1007c enables the user to know the days when ovulation may take place. Depending on the flow rate and other parameters the number of hygiene apparatus 110 required by the user may be enabled at 1009c. A share data and alert is enabled at 1011c. This function enables the user to share the data obtained with the health care provider for further analysis. The alerts can be shared with the respective family member to know the fertility window, the date of commencement of the next menstrual cycle and the number of apparatus 110 required to be utilized for a senior citizen and/or the frequency to change the apparatus 110.

Although the present disclosure has been described in terms of certain preferred embodiments and illustrations thereof, other embodiments and modifications to preferred embodiments may be possible that are within the principles and spirit of the invention. The above descriptions and figures are therefore to be regarded as illustrative and not restrictive.

Thus the scope of the present disclosure is defined by the appended claims and includes both combinations and sub combinations of the various features described herein above as well as variations and modifications thereof, which would occur to persons skilled in the art upon reading the foregoing description.

What is claimed is:

1. A system comprising:
an electronic module positioned in a multifunctional disposable hygiene apparatus of a user, wherein the electronic module comprises: a plurality of sensors enabled to sense a plurality of parameters of a discharge and read a pressure generated due to a seating pattern of the user; and an embedded dipstick configured to conduct a test of a plurality of blood parameters from the discharge;
a network device of a user comprising an application and in communication with the multifunctional disposable hygiene apparatus, wherein the application is configured to receive a data comprising data comprising the plurality of parameters of a discharge and pressure generated due to the seating pattern of the user and the plurality of blood parameters from the discharge from the electronic module;
a remote data centre in communication with the user's network device configured to store and process the data received from the user's network device and transmitting notifications to the user's network device in response to processing the data; and
a medical service provider's network device in communication with the user's data centre and the user's network device configured to allow the medical service provider to view the data of the user.

2. The system of claim 1, wherein the electronic module assigned with an electronic readable code for establishing a secured communication with the network device of the user.

3. The system of claim 1, wherein the dipstick enabled for a colour change as per the requisite test readings of a plurality of blood parameters from the discharge.

4. The system of claim 1, wherein the plurality of sensors further configured to sense at least one of: an odour of the discharge; a colour of the discharge; a temperature of the body of the user and the threshold limit of liquid absorbed.

5. The system of claim 1, wherein the electronic module configured to communicate with the application and alert the user at least one of: fullness of the multifunctional disposable hygiene apparatus.

6. The system of claim 1, wherein the remote data centre comprising an a data analysis and notification unit configured analyse data and transmit a plurality of notifications comprising at least one of: the number of days left for the next menstrual cycle to commence; total number of absorbent multifunctional disposable hygiene apparatus required depending on the pervious data; number of days of higher fertility; the days of lower pregnancy rate; and number of days when the ovulation is expected.

7. A method comprising:
pairing a multifunctional disposable hygiene apparatus with a user's network device by means of an electronic readable code of the electronic module positioned in the multifunctional disposable hygiene apparatus;
transmitting a data comprising data comprising a plurality of parameters of a discharge and pressure generated due to the seating pattern of the user and the plurality of blood parameters from the discharge from the electronic module to the user's network device configured with an application to receive the data;
storing and processing the data received from the user's network device and transmitting notifications to the user's network device in response to processing the data; and
transmitting notifications to the user's network device in response to processing the data by the remote data centre, wherein the application displays the notifications on a user interface of the user's network device.

8. The method of claim 7 comprising a step of enabling the user to enter the regularity of the cycle and entering the first date of the previous menstrual cycle along with the average number of days in the cycle.

9. The method of claim 7, wherein the application is configured to display a calendar view and a circular view of a current date, a projected date of a menstrual cycle and number of days remaining for the cycle to start.

10. The method of claim 7, wherein the application in communication with the electronic display configured to display at least one of: a degree of fullness due to a frequent absorption of the discharge; a flow rate of the discharge; a body temperature of the user; average number of days involved in the user's cycle; and a regularity and irregularity in the menstrual cycle.

11. The method of claim 7, wherein the application further configured to display readings of the data in a selected month in a graphical way.
